(19) United States
(12) Patent Application Publication
(10) Pub. No.: US 2021/0220368 A1
(43) Pub. Date: Jul. 22, 2021

(54) COMPOSITIONS COMPRISING TIMOLOL AND THEIR USE IN THE TREATMENT OF ROSACEA BY TOPICAL ADMINISTRATION

(71) Applicant: Almirall, S.A., Barcelona (ES)

(72) Inventors: Bernat Vidal Juan, Barcelona (ES); Maria Victoria Revilla Sanchez, Barcelona (ES); Gema Tarrason Encuentra, Barcelona (ES)

(73) Assignee: Almirall, S.A., Barcelona (ES)

(21) Appl. No.: 16/309,808

(22) PCT Filed: Jun. 15, 2017
(86) PCT No.: PCT/EP2017/064704

§ 371 Date: Dec. 13, 2018

(30) Foreign Application Priority Data
Jun. 16, 2016 (EP) 16382278.6

(51) Int. Cl. A61K 31/5377; A61P 17/00; A61K 9/00; A61K 9/06

(52) U.S. Cl. CPC A61K 31/5377; A61K 9/06; A61K 9/0014; A61P 17/00

(57) ABSTRACT

The present invention relates to the use of timolol in treating rosacea, and to pharmaceutical compositions comprising timolol.

The present invention relates to the use of timolol in treating rosacea, and to pharmaceutical compositions comprising timolol.

### BACKGROUND TO THE INVENTION

Rosacea is a common chronic-recurrent, usually symmetrical, facial dermatosis that persists for years with periods of exacerbation and remission. It is a chronic inflammatory cutaneous disease primarily affecting the central face of adults aged between 25 and 70. Rosacea can be categorized into four subtypes: (1) erythematotelangiectatic rosacea (ETR) defined by the presence of flushing and central facial erythema, (2) papulopustular rosacea (PPR) defined by the presence of persistent erythema and transient papules or pustules, (3) phymatous rosacea, presenting with thick skin, irregular surface nodularities, and enlargement of face skin surfaces such as the nose (rhinophyma), and (4) an ocular subtype that presents as dryness, irritation, blepharitis, conjunctivitis, or keratitis, and that can compromise eyesight.

Rosacea occurs both in men and women, although there are some gender differences. It usually starts earlier among females, whereas rhinophyma is almost exclusively seen among males. Rosacea is more frequent in patients with fair skin and conservative estimates suggest that the disease affects 14 million individuals in the US alone, at a prevalence of 5%. It has an impact on patients' quality of life, since their physical appearance negatively influences their social and emotional health.

Conventional treatments for rosacea have focused on the inflammatory lesions, pustules and papules. Typically anti-microbial metronidazole, azelaic acid or sodium sulfacetamide-sulphur topical formulations are used to treat subtype 2 (PPR). Oral tetracyclines, such as doxycyline and minocycline, are also widely used for systemic treatment in rosacea subtypes 2 and 4. Recently, modified release formulations of low dose doxycycline and minocycline have been developed to minimize gastrointestinal side effects and concern about antibiotic resistance. Also recently, topical ivermectin, an anti-helmintic drug, has been approved for the treatment of inflammatory lesions of rosacea.

Brimonidine tartrate, an agonist of the α₂ adrenergic receptors, in a gel formulation at 0.5% has been recently approved for the treatment of nontransient facial erythema acting on the cutaneous vascular component of the disease. However, brimonidine has been reported to induce transient worsening of the erythema and flushing in some patients, raising some concerns about its utility.

In spite of this varied range of oral and topical compounds, proper control of the disease, in particular of the most common clinical subtypes 1 and 2, has not yet been achieved.

Therefore, there have been attempts to treat rosacea by topical administration of drugs with different mechanisms of action, for example, with beta-adrenergic antagonists (beta-blockers). However, these attempts have been unsuccessful. Thus, Jaque et al., Rev. Chilena Dermatol, 2012, 28(4), pp. 418-430, reports a clinical study in which topical timolol was tested on human subjects suffering from erythematotelangiectatic rosacea with no clinical benefit.

It has now surprisingly been found that, contrary to the results reported in the art, topical timolol has efficacy in treating rosacea, in particular in treating the facial erythema which characterises the disease. The topical administration of timolol avoids the occurrence of the side-effects which would result from oral administration and is devoid of the rebound erythema effect caused by the treatment with alpha-1 or alpha-2 adrenergic receptor agonists like brimonidine.

### SUMMARY OF THE INVENTION

The present invention therefore provides timolol or a pharmaceutically acceptable salt thereof, for use in the topical treatment of rosacea.

The present invention also provides a topical pharmaceutical composition comprising timolol or a pharmaceutically acceptable salt thereof and a pharmaceutically acceptable carrier or diluent, for use in treating rosacea.

The present invention also provides a topical pharmaceutical composition comprising (a) timolol or a pharmaceutically acceptable salt thereof and (b) a pharmaceutically acceptable carrier or diluent, wherein the composition comprises an oil phase.

Also provided is use of timolol or a pharmaceutically acceptable salt thereof, or a pharmaceutical composition as defined above, in the manufacture of a medicament for the topical treatment of rosacea, and a method of treating rosacea in a patient, which method comprises topically administering to the patient timolol, or a pharmaceutically acceptable salt thereof, or a composition as defined above.

### BRIEF DESCRIPTION OF THE FIGURES

FIG. 1 is a comparison of the anti-oedema effect of brimonidine and timolol after two topical applications in the TPA-induced mouse ear oedema model.

FIG. 2 is a comparison of the erythema inhibition of timolol 1%, brimonidine 0.33% and oxymetazoline 0.88% after capsaicin-induced vasodilation.

### DETAILED DESCRIPTION OF THE INVENTION

Timolol is (S)-1-(tert-butylamino)-3-[(4-morpholin-4-yl-1,2,5-thiadiazol-3-yl)oxy]propan-2-ol. It has the structure:

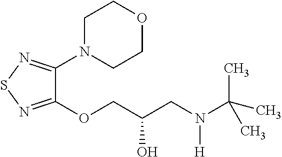 [SMILES: CC(C)(C)NC[C@H](O)COc1nsnc1N1CCOCC1]

The present invention provides timolol and pharmaceutical acceptable salts thereof for use in treating rosacea. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds for use in this invention include addition salts with a pharmaceutically acceptable acid such as such as hydrochloric acid, sulphuric acid, methanesulphonic acid, fumaric acid, maleic acid, succinic acid, acetic acid, benzoic acid, citric acid, tartaric acid or phosphoric acid. Other salts may be formed with a pharmaceutically acceptable base. Suitable such pharmaceutically acceptable salts include alkali metal salts, e.g. sodium or potassium salts; alkaline earth metal salts, e.g. calcium or magnesium salts; ammonium salts; and salts formed with suitable organic ligands, e.g. quaternary ammonium salts, and meglumine salts.

In a preferred embodiment of the invention the compound for use is timolol. In a further preferred embodiment of the invention the compound for use is timolol maleate.

Typically, the rosacea to be treated is erythematotelangiectatic rosacea or papulopustular rosacea. In another embodiment the rosacea to be treated is papulopostular rosacea, phymatous rosacea or rosacea subtype 4 (ocular rosacea). The compounds for use according to the invention are particularly useful in treating erythema and oedema caused by rosacea.

Typically the patient to be treated is a mammal. Preferably the patient is a human. More preferably the patient is a Caucasian human.

Typically, the timolol, the pharmaceutically acceptable salt thereof or the pharmaceutical composition of the present invention is applied topically to the face of a patient. Typically, it is not applied around the eyes. More typically it is not applied within 0.2 cm, more typically not within 0.5 cm, preferably not within 1 cm, of the eye.

Typically, the timolol, the pharmaceutically acceptable salt thereof or the pharmaceutical composition of the present invention is for use other than in conjunction with laser treatment, in particular Intense Pulsed Light (IPL) laser treatment. Thus, typically the patient treated according to the invention is not undergoing, and preferably has not been subjected to, such laser treatment.

Pharmaceutical compositions according to the invention are suitable for topical administration. Preferably, the compositions are suitable for topical administration but not suitable for ophthalmic administration.

For topical administration the pharmaceutical compositions of the present invention may take the form of any formulation normally used for topical administration, in particular solutions, lotions, emulsions of liquid consistency, emulsions of semi-liquid consistency, emulsions of semi-solid consistency, emulsions of solid consistency, creams, gels or ointments. Preferably the compositions of the present invention may take the form of a gel, a lotion or a cream; more preferably a lotion or a cream; still more preferably a cream.

The emulsions are obtained by dispersion of an oil phase in water (O/W) or a water phase in oil (W/O). Preferred pharmaceutical compositions for topical administration contain an oil phase. In a preferred embodiment, the pharmaceutical compositions of the present invention are water-in-oil emulsions (i.e. emulsions wherein the water is the dispersed phase and the oil in the dispersion medium). In another preferred embodiment, the pharmaceutical compositions of the present invention are oil-in-water emulsions (i.e. emulsions wherein the oil is the dispersed phase and the water in the dispersing medium).

Compositions for topical use in accordance with the invention may also contain one or more emollients, emulsifiers, thickeners and/or preservatives.

The emollients are typically long chain alcohols, such as cetyl alcohol, stearyl alcohol and cetearyl alcohol; hydrocarbons such as petrolatum and light mineral oil; or acetylated lanolin. The total amount of emollient in the formulation is preferably about 10% to about 20%, and more preferably about 5% to about 10% by weight based on the total weight of the formulation.

The emulsifier is typically a nonionic surface active agent, e.g., polysorbate 60 (available from Sigma Aldrich), sorbitan monostearate, polyglyceryl-4 oleate, and polyoxyethylene(4)lauryl ether or trivalent cationic. Generally the total amount of emulsifier is preferably about 2% to about 14%, and more preferably about 2% to about 6% by weight based on the total weight of the formulation.

Pharmaceutically acceptable thickeners, such as Veegum.TM.K (available from R. T. Vanderbilt Company, Inc.), and long chain alcohols (i.e. cetyl alcohol, stearyl alcohol or cetearyl alcohol) can be used. The total amount of thickener present is preferably about 3% to about 12% by weight based on the total weight of the formulation.

Preservatives such as methylparaben, propylparaben and benzyl alcohol can be present in the formulation.

Optionally, an additional solubilizing agent such as benzyl alcohol, lactic acid, acetic acid, stearic acid or hydrochloric acid can be included in the formulation.

Optionally, the formulation can contain a humectant such as glycerin and skin penetration enhancers such as butyl stearate.

It is known to those skilled in the art that a single ingredient can perform more than one function in a composition, i.e., cetyl alcohol can serve both as an emollient and as a thickener.

Preferably, the pharmaceutical composition of the invention comprises an oil phase.

Typically, the amount of oil in the composition is at least 10 wt. %, preferably at least 15 wt. %, more preferably at least 20 wt. %, based on the total weight of the composition. As used herein an oil phase is typically a liquid or solid phase which is substantially immiscible with water. More typically, an oil phase as used herein has a solubility in water at 25° C. of less than or equal to 1 mg/L, preferably less than 0.1 mg/L.

The oil phase in an emulsion may be any oil phase normally used in emulsions for topical administration. Such oil phases include, for example, hydrocarbon bases such as such as hard paraffin, soft paraffin, ceresine and microcrystalline wax, absorption bases such as lanolin and beeswax, emulsifying bases such as emulsifying wax and cetrimide, and vegetable oils such as olive oil, coconut oil, sesame oil, almond oil and peanut oil. Other oil phases useful in accordance with the invention are mineral oil, liquid petroleum, sorbitan monostearate, polysorbate 60, cetyl esters wax, cetearyl alcohol, benzyl alcohol and 2-octyldodecanol.

Those skilled in the art will understand that by varying the ratio of water to oil in an emulsion, the result could be deemed a lotion, a cream, or an ointment, by order of increasing proportion of oil. An emulsion comprising similar proportions of oil phase and water phase is usually deemed a cream, whereas an ointment will generally contain a substantially higher proportion of oil phase compared to water phase, for example greater than 60 wt. % oil phase, preferably greater than 70 wt. % oil phase, more preferably greater than 80 wt. % oil phase, based on the total weight of the oil phase and the water phase. A lotion will generally contain a lower proportion of oil phase than a cream, for example under 25 wt. % oil phase, under 20 wt. % oil phase, under 15 wt. % oil phase, under 10 wt. % oil phase or under 5 wt. % oil phase, based on the total weight of the oil phase and the water phase.

Generally, a cream for use according to the invention comprises an oil phase and a water phase mixed together to form an emulsion. Preferably, the amount of water present in a cream of the invention is about 45% to about 95% by weight based on the total weight of the cream, more preferably about 55 wt. % to about 90 wt. %, even more preferably about 65 wt. % to about 80 wt. %.

Where the composition is an ointment a pharmaceutically acceptable ointment base will be used. Examples of ointment bases include hydrocarbon bases such as such as hard paraffin, soft paraffin, ceresine and microcrystalline wax, absorption bases such as lanolin and beeswax, water-soluble bases such as polyethylene glycols (e.g. polyethylene glycol 200, 300, 400, 3350, 4000 or 6000), propylene glycol and polypropylene glycols, emulsifying bases such as emulsifying wax and cetrimide, and vegetable oils such as olive oil, coconut oil, sesame oil, almond oil and peanut oil. Mixtures of ointment bases can of course be used. The amount of ointment base present in an ointment of the invention is preferably about 60% to about 95% by weight based on the total weight of ointment, more preferably about 70 wt. % to about 90 wt. %, still more preferably about 75 wt. % to about 85 wt. %.

The pharmaceutical composition for use in accordance with the present invention may also be a lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

Typically, the pharmaceutical composition of the invention contains less than 90 wt. % water, preferably less than 80 wt. % water, based on the total weight of the composition.

Pharmaceutical compositions for use according to the present invention may be substantially non-aqueous. Typically, a substantially non-aqueous pharmaceutical composition comprises less than 25% water by weight, relative to the total weight of the composition, preferably less than 20%, more preferably less than 15%, even more preferably less than 10%, more preferably still less than 5%, still more preferably less than 2% and most preferably less than 1% water.

In the compositions for use according to the invention, the timolol or pharmaceutically acceptable salt thereof is present at a concentration of between 0.001 and 20% by weight (expressed as timolol free base), relative to the total weight of the composition, preferably between 0.01 and 10%, more preferably between 0.1 and 5% by weight, in particular 0.1%, 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75% or 5%. In a preferred embodiment, the timolol or pharmaceutically acceptable salt thereof is present at a concentration of 1% by weight (expressed as timolol free base), relative to the total weight of the composition.

In another preferred embodiment, the timolol or pharmaceutically acceptable salt thereof is present at a concentration of 0.5% by weight (expressed as timolol free base), relative to the total weight of the composition.

In another preferred embodiment, the timolol or pharmaceutically acceptable salt thereof is present at a concentration of 0.1% by weight (expressed as timolol free base), relative to the total weight of the composition.

The present invention is explained in more detail in the following by referring to the example below, which is not to be construed as limitative.

### Example 1

Evaluation of the Anti-Oedema Effect of Timolol and Brimonidine after Two Topical Applications in the TPA-Induced Ear Oedema Model in Balb/c Mice.

D. Piwnica et al. described in J. Dermatol. Sci., 75 (1) 49-54, 2014, that topical brimonidine 0.2% inhibited by 50% TPA-induced ear oedema in mice. According to these authors, this confirms that the “reduction of oedema in rosacea is a critical requirement for any new treatment”.

In view of these findings, the inventors of the present application used the same model to check whether timolol would be beneficial in the treatment of rosacea.

Experimental Design

The oedema is induced by means of a single application to the right ear of the mouse of 20 μl of a solution of TPA (phorbol 12-myristate 13-acetate) in acetone at 0.01%. The test compounds are diluted in acetone and applied 30 minutes before and 15 minutes after TPA. The weight of the mouse ears is measured at T+6 h and the weight of the left ear is subtracted from the one of the right ear.

Treatment

Timolol was applied at a concentration of 1% before and after TPA application. The alpha agonist, brimonidine 0.2% was also tested for comparison.

Results

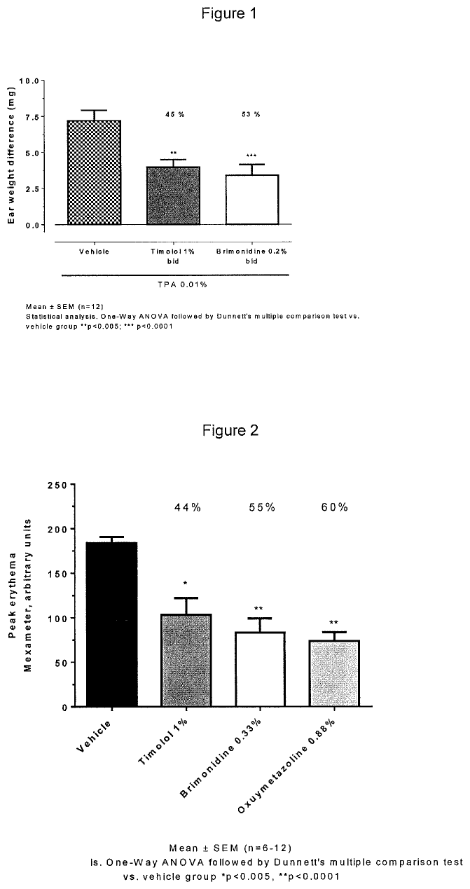

The results are shown in FIG. 1, which represents the average weight of the ear oedema in three groups of animals. Each group included 12 animals. Numbers above the bars indicate the percentage inhibition of the corresponding group of treated animals vs the control group. Both timolol and brimonidine produced a very significant inhibition (p<0.005) of ear oedema of around 50%.

These results show that timolol has an anti-oedema activity which is comparable to the one of the reference drug brimonidine. Timolol would be thus particularly beneficial in the treatment of rosacea, because, in addition to this anti-oedema activity, it would not produce a rebound effect.

### Example 2

Evaluation of the Effect of Topical Timolol, Brimonidine and Oxymetazoline in Vasodilation Induced by Topical Application of Capsaicin in the Ear of CD1 Mice

Dysregulation of innate and adaptive immune pathways as well as neuro-vascular changes are present in rosacea. A wide spectrum of “trigger factors” have been identified; physical such as UV or temperature, biological, including microbiota and food ingredients, and endogenous factors or stress. Rosacea disease kinetics with onset of flushing, erythema associated with somatosensory sensations, suggest a role for neuro-immune and neurovascular communications (Holmes & Steinhoff Exp Derm 2016).

Dermal neurogenic inflammation can occur after the topical application of capsaicin on the human skin. Capsaicin, activating TRPV1 channels in the skin, induces the release of pro-inflammatory neuropeptides, including Calcitonin gene related peptide (CGRP), which interacts with vascular smooth muscle cells and induces vasodilation in peripheral tissues (characterized by local redness and warmth). Topical application of capsaicin into the skin has been widely used to induce transient flare reactions and vascular dilatation increases in mice too (Buntinx et al. Br J Clin Pharm 2015). Effects can be monitored by laser Doppler or spectrophotometer analysis.

A similar model has been described in patent application WO2012001076(A1)1 to assess activity of medications for the treatment of rosacea.

Experimental Design

Neurogenic skin inflammation was induced by capsaicin (Alacapsin 0.075% cream) on the ear of CD-1 mice. Capsaicin induces the release of neuropeptides most of which have vasodilatory properties. In this model vasodilation is evaluated with a narrowband spectrophotometer probe (Mexameter) that measures peak absorption of haemoglobin. The quantity of light absorbed by the skin is calculated as Mexameter® arbitrary units, and this measure of erythema can be considered a surrogate of vasodilation. Maximal vasodilation response is achieved 45 minutes after capsaicin application. Treatment effects are reported at this maximal induction of erythema.

Treatment

Timolol 1%, brimonidine 0.33% and oxymetazoline 0.88% solutions were applied topically to the mouse ear before capsaicin induction.

Results

FIG. 2 shows the results of erythema inhibition by timolol 1%, brimonidine 0.33% and oxymetazoline 0.88% after capsaicin-induced vasodilation. Results described represent peak erythema of four groups of animals and are reported as mexameter arbitrary units. Each group included 6-12 animals. Numbers above the bars indicate the percentage inhibition of the corresponding group vs the capsaicin control group. All adrenergic drugs inhibited ear vasodilation induced by capsaicin, by 44%, 56% and 60% respectively. All three drugs showed statistically significant inhibition of erythema vs control (**p<0.0001 and *p<0.005). No statistically significant differences were observed among the 3 treatment groups of timolol, brimonidine and oxymetazoline.

These results show that timolol is able to inhibit erythema induced by neurogenic inflammation similar to approved drugs for the treatment of persistent erythema of rosacea.

1. A method of treating rosacea in a patient, which method comprises administering topically to the patient a compound which is timolol or a pharmaceutically acceptable salt thereof.

2. The method according to claim 1, wherein timolol is in the form of timolol maleate.

3. A method of treating rosacea in a patient, the method comprising administering topically to the patient a pharmaceutical composition for topical administration comprising (a) timolol or a pharmaceutically acceptable salt thereof and (b) a pharmaceutically acceptable carrier or diluent.

4. The method according to claim 3, wherein the composition is a gel, a lotion or a cream.

5. The method according to claim 3, wherein timolol is in the form of timolol maleate.

6. A pharmaceutical composition for topical administration comprising (a) timolol or a pharmaceutically acceptable salt thereof and (b) a pharmaceutically acceptable carrier or diluent wherein the composition comprises an oil phase.

7. The pharmaceutical composition according to claim 6 which contains less than 90 wt. % water, based on the total weight of the composition.

8. The pharmaceutical composition according to claim 6, wherein the composition is a gel, a lotion or a cream.

9. The pharmaceutical composition according to claim 6 wherein timolol is in the form of timolol maleate.

10. The method according to claim 3, wherein the pharmaceutical composition comprises (a) timolol or a pharmaceutically acceptable salt thereof and (b) a pharmaceutically acceptable carrier or diluent, and wherein the composition comprises an oil phase.

11-12. (canceled)
